(19) United States
(12) Patent Application Publication
(10) Pub. No.: US 2010/0068221 A1
(43) Pub. Date: Mar. 18, 2010

(54) LOW SALT FOOD WITH IMPROVED TASTE

(76) Inventors: Arie Gerrit Terdu, Strijen (NL); Leendert Nicolaas Fontijne, Schiedam (NL); Jan Dirk Rene Hille, Wouw (NL); José Mastenbroek, 's-Gravenhage (NL); Bertus Noordam, 's-Gravenzande (NL)

Correspondence Address: NIXON & VANDERHYE, PC, 901 NORTH GLEBE ROAD, 11TH FLOOR, ARLINGTON, VA, 22203, US

(21) Appl. No.: 12/516,816

(22) PCT Filed: Nov. 26, 2007
(86) PCT No.: PCT/EP07/62801

§ 371 Date: Nov. 10, 2009

(30) Foreign Application Priority Data
Dec. 7, 2006 (EP) 06125590.7
Jun. 4, 2007 (EP) 071209501.2

(51) Int. Cl. A61K 36/06; A21D 8/04; A21D 2/00; A23L 1/30; A61P 9/12; A61P 9/00

(52) U.S. Cl. 424/195.16; 426/18

(57) ABSTRACT

The present invention relates to the use of a yeast extract for preparing a cereal product with a reduced sodium. The yeast extract comprises at least 30% w/w 5′-ribonucleotides on the basis of sodium chloride free dry matter. Preferably, a yeast extract is used wherein the total amount of 5′-GMP plus 5′-IMP is at least 15% w/w on the basis of sodium chloride free dry matter. The method of the invention may advantageously be used for preparing a reduced sodium cereal products wherein at least 30% of the sodium is replaced, since off-tastes are masked and salt perception is enhanced.

### FIELD OF THE INVENTION

The present invention relates to the use of a yeast extract for the improvement of taste in cereal products. In particular, it relates to the improvement of taste of low-sodium cereal products with reduced sodium content.

### BACKGROUND OF THE INVENTION

The use of sodium chloride, has long been suspected as a cause of health problems, particularly hypertension. It is very difficult to limit salt intake to very low levels because processed foods, takeaway foods and restaurant-prepared foods typically contain large amounts of salt. At the same time, there is a trend with many food processors to reduce the amount of sodium in processed food, in particular in cereal products because they constitute a large part of our daily diet. For example, bread contains a substantial amount of sodium chloride and is consumed every day. To some degree the problem of high salt levels in foods, such as bread, could be reduced by simply lowering the level of salt added to the food, however this leads to a reduced salt perception and is therefore undesired. Another disadvantage of salt substitutes, such as potassium chloride, is that they impart an “off” flavor to food. As a consequence, it is necessary to use additives to mask the off-flavor of the salt substitutes.

U.S. Pat. No. 4,297,375 discloses the use of an autolyzed yeast to reduce bitterness in foods caused by the presence of potassium chloride-containing salt substitutes.

EP0103994 describes a sodium chloride substitute flavoring system which comprises autolyzed yeast and ammonium chloride. The system improves the saltiness of foods, reduces the bitterness of sodium chloride substitutes and is suitable for use in a sodium chloride substitute system for savoury applications.

WO 2005/087013 describes the use of a dry taste enhancing agent for cereal products which taste-enhancing agent comprises a yeast extract and acid fermented flour.

A problem associated with the yeast extracts as generally described in the prior art is that they can cause the food to which they are added to get a bouillon-like, brothy taste or after taste associated with yeast (yeasty taste and/or odour). This problem is especially evident in beverages and in non-savoury food.

### DETAILED DESCRIPTION OF THE INVENTION

The present invention relates to the use of a yeast extract for preparing a cereal product with reduced sodium content compared to a corresponding conventional cereal product, wherein the yeast extract comprises at least 30% w/w 5′-ribonucleotides on the basis of sodium chloride free dry matter. This may lead to a reduced sodium, low-sodium, a very low sodium or a sodium-free cereal product.

There are several advantages of using such yeast extract for preparing cereal products with reduced sodium content. One advantage is that bitter off-tastes, for instance from salt substitutes such as potassium chloride, are masked if a yeast extract comprising at least 30% w/w 5′-ribonucleotides on the basis of sodium chloride free dry matter is added to the flour for low-sodium cereal products.

Another advantage is that the yeast extract effects a neutral taste enhancement, i.e. taste enhancement without the introduction of a brothy, bouillon note or yeasty note.

Yet another advantage is that by using a yeast extract which comprises at least 30% w/w 5′-ribonucleotides on the basis of sodium chloride free dry matter the salt perception is enhanced. This is of course very advantageous in reduced-sodium food applications. As a result, less sodium and less sodium replacers or taste enhancers are necessary to improve the taste of a reduced-sodium cereal product.

Another advantage is that there is no requirement to label cereal products which have been prepared according to the invention as containing chemical additives.

The term “cereal product with reduced sodium” refers to a cereal product wherein the amount of sodium in the product is less than the amount of sodium conventionally present in the corresponding conventional cereal product. Preferably the cereal product will comprise at least 25% w/w less sodium than conventionally present in the corresponding conventional cereal product. Said cereal product with reduced amount of sodium may comprise at least 25% w/w, 30% w/w, 35% w/w or 40% w/w less sodium, more preferably at least 45% w/w, 50% w/w, 55% w/w or 60% w/w less sodium than conventionally present in the corresponding conventional cereal product. The cereal product with reduced sodium prepared according to the method of the first aspect may comprise up to ˜100% w/w less sodium than conventionally present in the corresponding conventional cereal product.

The amount of sodium conventionally present in the cereal product is the amount present when the cereal product has not been altered, processed or formulated in order to reduce its sodium content and will depend on the type of cereal product, hence the reference to a ‘corresponding’ cereal product. Those skilled in the art know the amount of sodium which is conventionally present in a cereal product depending on the type of cereal product.

According to the invention, a yeast extract is used which comprises at least 30% w/w 5′-ribonucleotides, preferably at least 34% w/w, 38% w/w, 40% w/w or 42% w/w, more preferably at least 44% w/w, 46% w/w, 48% w/w or at least 50% w/w 5′-ribonucleotides on the basis of sodium chloride free dry matter.

In the context of the present invention, the phrase “5′-ribonucleotides” refers to the total amount of 5′-monophosphate ribonucleotides formed during RNA degradation, viz. 5′-monophosphate guanine (5′-GMP), 5′-monophosphate uracil (5′-UMP), 5′-monophosphate cytosine (5′-CMP), 5′-monophosphate adenine (5′-AMP), where 5′-AMP may be partially or completely converted into 5′-monophosphate inosine (5′-IMP). For example, in a yeast extract which comprises 30% w/w 5′-ribonucleotides on the basis of sodium chloride free dry matter, the total amount of 5′-GMP, 5′-UMP, 5′-CMP, 5′-AMP and 5′-IMP is 30% w/w on the basis of sodium chloride free dry matter.

In a preferred embodiment, a yeast extract is used wherein the total amount of 5′-GMP plus 5′-IMP is at least 15% w/w, preferably at least 17% w/w, 19% w/w, 20% w/w or 21% w/w, more preferably at least 22% w/w, 23% w/w, 24% w/w or 25% w/w, on the basis of sodium chloride free dry matter. Due to the constitution of RNA, from which the 5′-ribonucleotides arise, 5′-GMP and 5′-IMP will always be present in approximately equal amounts in this embodiment.

In the context of the present invention, weight percentage calculations of the 5′-ribonucleotides are based on the disodium salt heptahydrate thereof unless otherwise specified. All percentages are calculated on sodium chloride free dry matter (sfdm). In the present invention, the phrase ‘sodium chloride free dry matter (sfdm)’ refers to the fact that for the calculation of the weight percentage the weight of any sodium chloride present in the yeast extract is excluded from the composition. The measurement of sodium chloride in the yeast extract and the above-mentioned calculation can be performed by methods known to those skilled in the art.

The yeast extract may be prepared by any method which yields a yeast extract which comprises at least 30% w/w 5′-ribonucleotides (sfdm).

In one embodiment of the invention, the yeast extract is obtained by hydrolysis. Hydrolytic yeast extracts are concentrates of the soluble material obtained from yeast after disruption of the cells, digestion (lysis) and addition of exogenous enzymes such as proteases and/or peptidases and especially nucleases, such as 5′-phosphodiesterase and optionally 5′-adenylic deaminase, to the yeast suspension during lysis. The native yeast enzymes are generally inactivated prior to the lysis. During this process 5′-GMP, 5′-UMP, 5′-CMP and 5′-AMP may be formed. When adenylic deaminase is added to the mixture, 5′-AMP is transformed into 5′-IMP. Methods to produce hydrolytic yeast extracts are known in the art, see for example WO88/05267.

In another embodiment, the yeast extract is obtained by autolysis, for instance as described in WO2005/067734.

Preferably, the 5′-ribonucleotides of the yeast extract are produced in the autolytic or hydrolytic extraction process, i.e. in a natural process, as described in the previous paragraph. However, the skilled person will understand that a yeast extract which comprises at least 30% w/w 5′-ribonucleotides (sfdm) may be obtained by adding isolated or chemically synthesized 5′-ribonucleotides to a yeast extract which contains less than 30% w/w 5′-ribonucleotides. The use of these types of yeast extract is also encompassed in the present invention, but is less preferred. It has the disadvantage that food products prepared with such yeast extracts will have to be labeled as containing chemical additives.

With the methods mentioned above also yeast extracts may be produced which do not fulfil the requirements of the first aspect of the invention. However the yeast extracts comprising the proper amount of 5′-ribonucleotides may be selected by measuring the amounts thereof using methods known to those skilled in the art and by selecting those yeast extracts which comprises at least 30% w/w 5′-ribonucleotides (sfdm).

According to the invention, yeast extracts may be used which comprise up to 90% w/w or even 100% w/w 5′-ribonucleotides on the basis of sodium chloride free dry matter, although such compositions are not yeast extracts as intended by the Food Chemical Codex.

In another aspect, the present invention relates to a method for preparing a cereal product with reduced sodium content compared to a corresponding conventional cereal product, from a flour-based dough, wherein the method comprises replacing part of the sodium which is conventionally present in the dough by a yeast extract, wherein the yeast extract comprises at least 30% w/w 5′-ribonucleotides (sfdm). In a preferred embodiment, the reduced-sodium cereal product is prepared using a yeast extract which comprises at least 15% w/w of 5′-GMP plus 5′-IMP on the basis of sodium chloride free dry matter. By this method a reduced-sodium cereal product, or the intermediate product, may be obtained which is fully acceptable to the person skilled in the art. For example, a bread with reduced sodium content compared to a corresponding conventional bread may be obtained which has good volume, crumb structure and softness.

The yeast extracts used in the context of the invention may be obtained from any yeast. Suitable yeast strains which may be used belong to the genera Saccharomyces, Kluyveromyces and Candida. Yeast strains belonging to the genus Saccharomyces, for example to the species Saccharomyces cerevisiae, are preferred.

Methods for preparing a baked cereal product are known in the art, e.g. from Baking Science & Technology (1988, 3rd edition) ed. P. J. Pyler, Sosland Publishing Co, Kansas City, Miss., USA and typically include preparing, kneading, fermentation and baking of a dough. Baked cereal products are typically prepared from a flour-based dough and sodium chloride is invariably one of the constituents of the dough. Suitable examples of cereal and in particular of baked cereal products include all types of bread, such as white bread, sliced bread, soda bread, brown bread, wholemeal bread, malt bread, granary bread, rye bread, corn bread, pitta bread, baguettes, French sticks, bagels, rolls, croissants, pan bread; all types of cakes and cookies, such as muffins, scones, pound cake, sponge cake, pancake, brownies, biscuits, crisps, short bread, crackers, fruit cake; and also including pappadums, chapatti, nan, paratta, polenta, taco, tortilla, corn flakes, croutons and any type of pasta.

Therefore, in one embodiment of the present invention, a yeast extract which comprises at least 30% w/w 5′-ribonucleotides (sfdm) is used to prepare a reduced-sodium baked cereal product, i.e. a baked cereal product which is produced form a dough which comprises less sodium than conventionally used in doughs for baked cereal products. In a preferred embodiment, a yeast extract comprising at least 15% w/w 5′-GMP plus 5′-IMP is used to prepare a reduced-sodium baked cereal product.

All or part of the sodium in the dough may be replaced by yeast extract which comprises at least 30% w/w 5′-ribonucleotides or at least 15% w/w of 5′-GMP plus 5′-IMP (sfdm). Preferably, the dough used for preparing the cereal product comprises at least 25%, 30%, 35% or 40% w/w less sodium, more preferably at least 45%, 50%, 55% or 60% w/w less sodium than conventionally used. The amount of sodium conventionally present in a cereal product is the amount present when the cereal product has not been altered, processed or formulated in order to reduce its sodium content and will depend on the type of cereal product, hence the reference to a ‘corresponding’ cereal product. Those skilled in the art know the amount of sodium which is conventionally present in a certain type of cereal product. The sodium is typically present in the form of sodium chloride (NaCl).

Therefore, in another aspect, the invention relates to a cereal product with reduced amounts of sodium in comparison to a corresponding conventional cereal product, prepared from a flour and sodium-containing dough which dough comprises (i) less sodium than the corresponding conventional cereal product and (ii) 0.01% to 2% w/w of a yeast extract which comprises at least 30% w/w 5′-ribonucleotides (sfdm). The reduced-sodium cereal product is prepared from a flour and sodium-containing dough which dough comprises (i) preferably at least 25%, 30%, 35% or 40% w/w less sodium, more preferably at least 45%, 50%, 55% or 60% w/w less sodium than the corresponding conventional cereal product and (ii) preferably 0.01% to 2% w/w, more preferably 0.05% to 1% w/w of a yeast extract which comprises at least 30% w/w 5′-ribonucleotides (sfdm). Depending on the amount of sodium left in the product and national legislation, such reduced-sodium products may also be referred to as low-sodium, very low sodium or sodium-free cereal products. In a preferred embodiment, the yeast extract is used to prepare a reduced-sodium bread product. In one embodiment, 0.01% to 2% w/w yeast extract on the total weight of flour is used. Preferably, 0.05% to 1% w/w yeast extract on the total weight of flour is used for preparing a reduced-sodium bread.

The yeast extract which comprises at least 30% w/w 5′-ribonucleotides or at least 15% w/w of 5′-GMP plus 5′-IMP (sfdm) may be used in combination with other salt substitutes and salt taste enhancers known in the art, such as LiCl, LiBr, KCl, NH₄Cl, other ammonium salts, dipeptides and amino acids derivatives. Therefore, in another aspect the invention relates to a blend suitable for preparing a reduced-sodium cereal product. In one embodiment, the blend comprises a yeast extract which comprises at least 30% w/w 5′-ribonucleotides (sfdm) in combination with KCl, and preferably NH₄Cl. Suitable amounts are 30-70%, preferably 40-60%, more preferably 50-60% w/w KCl; 5-30%, preferably 10-20%, more preferably 15-20% w/w NH₄Cl; and 5-35%, preferably 10-30%, more preferably 12-25% w/w of a yeast extract which comprises at least 30% w/w 5′-ribonucleotides (sfdm). Less than 1% w/w on flour weight, typically, 0.1-0.8% w/w on flour weight of such blends is used for preparing reduced-sodium cereal products. A carrier may also be included. If a carrier is used, the carrier is preferably an oil, more preferably a vegetable oil. Preferably, 0.1-1% w/w of the carrier is used. All the products and blends of the present invention may advantageously be used in a low-sodium, reduced-sodium or no-sodium diet or in the treatment of hypertension or cardiovascular disease.

### EXAMPLES

### Example 1

### The Effect of a 5′-Ribonucleotide-Rich Yeast Extract on the Taste of a Bread with Reduced Sodium Content

In this example the effect of a 5′-ribonucleotide-rich yeast extract on the taste of a bread with reduced sodium is demonstrated.

### Procedure:

### Ingredients:

5′-ribonucleotide-rich yeast extract containing 20% w/w 5′GMP+5′IMP, 40% w/w 5′-ribonucleotides and <1% sodium chloride on dry matter, from DSM Food Specialties, the Netherlands.

Fungal α-amylase from DSM Food Specialties, the Netherlands.

Hemi-cellulase from DSM Food Specialties, the Netherlands.

Bread dough was prepared according to the scheme presented in Table 1. In all tests including the reference 70 g compressed yeast (2% w/w based on total weight) were also present in the bread ingredients.

TABLE 1
dough composition

| Ingredient | Reference | Test 1 | Test 2 | Test 3 | Test 4 |
|---|---|---|---|---|---|
| Flour (g) | 3500 | 3500 | 3500 | 3500 | 3500 |
| Sodium chloride (g) | 70 | 48 | 48 | 48 | 48 |
| KCl (g) | — | — | 9.6 | 9.6 | — |
| NH₄Cl (g) | — | — | 3.0 | 3.0 | — |
| Ascorbic acid (mg) | 140 | 140 | 140 | 140 | 140 |
| Fungal alpha- | 10.5 | 10.5 | 10.5 | 10.5 | 10.5 |
| amylase (mg) | | | | | |
| Hemicellulase (mg) | 52.5 | 52.5 | 52.5 | 52.5 | 52.5 |
| Water (g) | 1972 | 1972 | 1972 | 1972 | 1972 |
| Yeast extract with | — | — | — | 3.5 | 3.5 |
| 20% w/w 5′-GMP | | | | | |
| plus 5′-IMP (sfdm) (g) | | | | | |

The ingredients were mixed into a dough using a Kemper® spiral mixer for 2 minutes at speed 1 followed by 6 minutes mixing at speed 2. Dough temperature was 28° C. Bulk proof was 5 minutes at 34° C./85% relative humidity. Next, dough pieces of 875 g were rounded, proofed for 35 minutes at 34° C./85% relative humidity. Finally, dough pieces were punched, moulded, panned, proofed for 75 minutes at 34° C./85% relative humidity and baked for 20 minutes at 220° C.

Dough properties (Table 2) and the breads produced from the different doughs were evaluated. Taste was evaluated by an experienced taste panel and compared with the taste of the reference bread (Table 3). Special attention was paid if bouillon notes could be tasted in the bread which contained the yeast extract, because bouillon notes are normally associated with the application of yeast extract.

TABLE 2
dough properties of the different tests

| Dough attribute | Reference | Test 1 | Test 2 | Test 3 | Test 4 |
|---|---|---|---|---|---|
| Consistency | 0 | 0 | 0 | 0 | −1 |
| Extensibility | 0 | 1 | 1 | 1 | −1 |
| Development/body | 1 | 0 | 0 | 1 | 1 |
| Stickiness | 0 | 0 | 0 | 0 | 1 |

Scores within −3 and 3, reference = 0, +/−1 = minor deviation from reference, fully acceptable, +/−2 = major deviation from reference, in most cases not acceptable, +/−3 = major deviation, absolutely not acceptable.

From Table 2 it is clear that 30% salt reduction results in less developed and more extensible dough. Addition of small quantities of KCl/NH4Cl does not really change dough rheology. After addition of the 5′-ribonucleotide-rich yeast extract, dough development was similar to the reference, only extensibility was somewhat increased. This difference to the reference is fully acceptable to the skilled person.

After baking, no significant differences were seen in loaf volumes.

The dough properties of each test were comparable to that of the reference dough. The final breads were evaluated on taste by a trained taste panel. Results are presented in Table 3.

TABLE 3

| Taste attribute | Reference | Test 1 | Test 2 | Test 3 | Test 4 |
|---|---|---|---|---|---|
| Salt | 100 | 40 | 70 | 90 | 65 |
| Bitter | No | No | Yes | No | No |
| Bouillon/yeasty | No | No | No | No | No |
| off-taste | | | | | |

The results in Table 3 clearly demonstrate that the salt perception in bread having 30% less sodium chloride can be compensated almost completely with a yeast extract which comprises at least 15% w/w of 5′-GMP plus 5′-IMP (and thus at least 30% w/w 5′-ribonucleotides (sfdm)). If such yeast extract is added to a dough which also comprises potassium chloride and ammonium chloride, the yeast extract will also mask the bitter off-taste introduced with the potassium chloride. The fact that no bouillon-like note was tasted in bread of Test 3 or 4 makes it clear that the background- or bouillon taste normally present in yeast extract is almost absent.

### Example 2

### The Effect of the 5′-Ribonucleotide Concentration of Yeast Extract on the Taste of a Bread with Reduced Sodium Content

This example was done to demonstrate the effect of the 5′-ribonucleotide concentration on taste of bread with reduced amount of sodium chloride.

### Procedure:

### Ingredients:

5′-ribonucleotide-rich yeast extract containing 20% w/w 5′GMP+5′IMP, 40% w/w 5′-ribonucleotides and <1% sodium chloride on dry matter, from DSM Food Specialties, the Netherlands.

5′-ribonucleotide containing yeast extract containing 11.2% w/w 5′GMP+5′IMP, 22.4% w/w 5′-ribonucleotides and 20% of sodium chloride on dry matter (DSM Food Specialties). This is equivalent to 14% w/w 5′GMP+5′IMP and 28% w/w 5′-ribonucleotides on sodium chloride free dry matter basis.

Fungal alpha-amylase from DSM Food Specialties, the Netherlands.

Hemi-cellulase from DSM Food Specialties, the Netherlands.

Both yeast extracts were dosed on 5′-ribonucleotide content.

Bread dough has been prepared according to the scheme presented in Table 4. In all tests including the reference 70 g compressed yeast (2% w/w based on total weight) were also present in the bread ingredients.

TABLE 4
dough composition

| Ingredient | Reference | Test 1 | Test 2 | |
|---|---|---|---|---|
| Flour | 3500 | 3500 | 3500 | g |
| Sodium chloride | 70 | 46.7* | 48 | g |
| KCl | — | 9.6 | 9.6 | g |
| NH₄Cl | — | 3.0 | 3.0 | g |
| Ascorbic acid | 140 | 140 | 140 | mg |
| Fungal alpha-amylase | 10.5 | 10.5 | 10.5 | mg |
| Hemi cellulase | 52.5 | 52.5 | 52.5 | mg |
| Water | 1972 | 1972 | 1972 | g |
| Yeast extract containing | — | 6.3 | — | g |
| 14% w/w 5′-GMP plus | | | | |
| 5′-IMP (sfdm) | | | | |
| Yeast extract containing | — | — | 3.5 | g |
| 20% w/w 5′-GMP plus | | | | |
| 5′-IMP (sfdm) | | | | |

*Compensated because of the sodium chloride content in this yeast extract sample

The ingredients were mixed into a dough using a Kemper® spiral mixer for 2 minutes at speed 1 followed by 6 minutes mixing at speed 2. Dough temperature was 28° C. Bulk proof was 5 minutes at 34° C./85% relative humidity. Next, dough pieces of 875 g were rounded, proofed for 35 minutes at 34° C./85% relative humidity. Finally, dough pieces were punched, moulded, panned, proofed for 75 minutes at 34° C./85% RH and baked for 20 minutes at 220° C.

Dough properties (Table 5) and the breads produced from the different doughs were evaluated. Taste was evaluated by an experienced taste panel and compared with the taste of the reference bread (Table 6). Special attention was paid if bouillon notes could be tasted in the bread enriched with the yeast extract, because bouillon notes are normally associated with the application of yeast extract.

TABLE 5
dough properties of the different tests

| Dough attribute | Reference | Test 1 | Test 2 |
|---|---|---|---|
| Consistency | 0 | 1 | −1 |
| Extensibility | 0 | 2 | 1 |
| Development/body | 1 | 2 | 1 |
| Stickiness | 1 | 2 | 0 |

Scores within −3 and 3, reference = 0, +/−1 = minor deviation from reference, fully acceptable, +/−2 = major deviation from reference, in most cases not acceptable, +/−3 = major deviation, absolutely not acceptable.

The results demonstrate that the extensibility changed too much for the dough containing the yeast extract which comprised 14% w/w 5′-GMP plus 5′-IMP. In addition, small differences were noticed on the consistency, dough development and stickiness. Especially, the difference in extensibility made the dough properties unacceptable. Addition of the yeast extract containing 20% w/w 5′-GMP plus 5′-IMP resulted only in small differences on the consistency, extensibility and stickiness. These differences to the reference are fully acceptable to the skilled person.

The final breads were evaluated on taste by a trained taste panel. Results are presented in Table 6.

TABLE 6

| Dough type | Taste result of the bread |
|---|---|
| Test 1 | The bread had an awful cardboard and a slightly bouillon- |
| | like taste. |
| Test 2 | Salt perception almost as reference, no bitter off-taste, |
| | no bouillon-like taste. |

The results presented in Table 6 clearly demonstrate the good results obtained if yeast extract is used which comprises at least 15% w/w of 5′-GMP plus 5′-IMP (and thus at least 30% w/w 5′-ribonucleotides (sfdm)) on the basis of sodium chloride free dry matter.

1. Use of a yeast extract for preparing a cereal product with a reduced sodium content in comparison to a corresponding conventional cereal product, wherein the yeast extract comprises at least 30% w/w 5′-ribonucleotides on the basis of sodium chloride free dry matter.

2. Use of a yeast extract according to claim 1, wherein the yeast extract comprises at least 15% w/w of 5′-GMP plus 5′-IMP on the basis of sodium chloride free dry matter.

3. Use according to claim 2 wherein the yeast extract contains at least 20% w/w 5′-GMP plus 5′-IMP on the basis of sodium chloride free dry matter.

4. Use according to claim 1 wherein the 5′-nucleotides are produced in an autolytic or hydrolytic extraction process.

5. Use according to claim 1 wherein the yeast extract is prepared from Saccharomyces yeast.

6. Use according to claim 1 wherein the cereal product is a baked cereal product, preferably bread.

7. Method for preparing a cereal product with a reduced sodium content in comparison to a corresponding conventional cereal product, from a flour-based and sodium-containing dough wherein the method comprises replacing part of the sodium which is conventionally present in the dough by a yeast extract, wherein the yeast extract comprises at least 30% w/w 5′-ribonucleotides on the basis of sodium chloride free dry matter.

8. Method according to claim 7 wherein the method further comprises adding a further salt substitute or further taste enhancer to the flour.

9. Method according to claim 7 wherein the cereal product with a reduced sodium content in comparison to a corresponding conventional cereal product, is prepared from a dough which contains at least 30% less sodium than a dough for the corresponding conventional cereal product.

10. Method according to claim 9 wherein the cereal product with a reduced sodium content in comparison to a corresponding conventional cereal product, is prepared from a dough which contains at least 50% less sodium than a dough for the corresponding conventional cereal product.

11. Method according to claim 10 wherein the cereal product is a baked cereal product, preferably bread.

12. Cereal product with a reduced sodium content in comparison to a corresponding conventional cereal product, prepared from a flour-based and sodium-containing dough which dough comprises (i) at least 30% w/w less sodium than the corresponding conventional cereal product and (ii) 0.01% to 2% w/w of a yeast extract which comprises at least 30% w/w 5′-ribonucleotides on the basis of sodium chloride free dry matter.

13. A blend for preparing a cereal product with a reduced sodium content in comparison to a corresponding conventional cereal product, which blend comprises:
(a) 30-70% w/w KCl
(b) 5-30% w/w NH₄Cl
(c) 5-35% w/w of a yeast extract which comprises at least 30% w/w 5′-ribonucleotides on the basis of sodium chloride free dry matter.

14. Use of a yeast extract which comprises at least 30% w/w 5′-ribonucleotides on the basis of sodium chloride free dry matter, of a reduced sodium cereal product according to claim 12 in a reduced-sodium, no-sodium or low-sodium diet.

15. Use of a yeast extract which comprises at least 30% w/w 5′-ribonucleotides on the basis of sodium chloride free dry matter for the preparation of a food with reduced sodium for use in a reduced-sodium, no-sodium or low-sodium diet or in the treatment of hypertension or cardiovascular disease.

16. Use according to claim 15, wherein the food is a cereal product.

17. A yeast extract which comprises at least 30% w/w 5′-ribonucleotides on the basis of sodium chloride free dry matter for use in a reduced-sodium, no-sodium or low-sodium diet or for use in the treatment of hypertension or cardiovascular disease
